(19) United States
(12) Patent Application Publication
(10) Pub. No.: US 2025/0082314 A1
(43) Pub. Date: Mar. 13, 2025

(54) HANDLE FOR MEDICAL ENDOSCOPES, AND ENDOSCOPE SYSTEM

(71) Applicant: Joimax GmbH, Karlsruhe (DE)

(72) Inventors: Christoph HOFSTETTER, Seattle, WA (US); Justin BIRKNER, Biberach (DE)

(73) Assignee: Joimax GmbH, Karlsruhe (DE)

(21) Appl. No.: 18/958,747

(22) Filed: Nov. 25, 2024

Related U.S. Application Data
(63) Continuation of application No. PCT/EP2023/063733, filed on May 23, 2023.

(30) Foreign Application Priority Data
May 24, 2022 (DE) 10 2022 113 124.6

(51) Int. Cl. A61B 17/00

(52) U.S. Cl. CPC A61B 17/00234; A61B 2017/0034; A61B 2017/00424; A61B 2017/00464

(57) ABSTRACT

A handle for medical endoscopes, wherein the handle has a proximal contact surface for the endoscope and a distal contact surface for the endoscope. The direction of extension of the proximal contact surface is oriented at an angle greater than 0° with respect to the direction of extension of the distal contact surface. The invention also relates to an endoscope system with a medical endoscope and a handle according to the invention, wherein the handle is connected to the endoscope.

This nonprovisional application is a continuation of International Application No. PCT/EP2023/063733, which was filed on May 23, 2023, and which claims priority to German Patent Application No. 10 2022 113 124.6, which was filed in Germany on May 24, 2022, and which are both herein incorporated by reference.

### BACKGROUND OF THE INVENTION

### Field of the Invention

The present invention relates to a handle for medical endoscopes and to an endoscope system.

### Description of the Background Art

The use of endoscopes during surgical procedures enables a surgeon to visually inspect surgical sites that are not visible to the naked eye. In addition, endoscopes enable the visual inspection of desired, otherwise invisible, areas of the body as part of a variety of diagnostic procedures. Minimally invasive surgical procedures would be unthinkable without the use of endoscopes for visual inspection of the surgical procedure and results, due to the necessarily small openings in the skin required for this purpose as access to the surgical site.

Typically, the endoscope is held by the hand of the surgeon or an assistant during a procedure and adjusted as necessary to allow the surgeon to visually inspect the surgical site throughout the procedure. However, especially in more complex procedures, this inevitably leads to fatigue when holding and adjusting the endoscope, which can impair the course of the procedure. For example, during orthopedic procedures on the spine, where access is created to an intervertebral area between two adjacent vertebral bodies, strict care must be taken to ensure that under no circumstances are nerves located near the spine damaged. This risk increases with the duration of the respective procedure, also due to the fatigue caused by holding and adjusting the endoscope.

To support the endoscopist, holding arms that can be freely moved in space are known from the prior art, which arms can rigidly hold endoscopes and thus allow the endoscope to be fixed in space at a largely arbitrary position. This means that the surgeon no longer has to hold the endoscope with his hand. The disadvantage here, however, is that, in order to readjust the endoscope, the surgeon has to realign it together with the holding device during the procedure. This is not merely mechanically complex. Especially during the aforementioned spinal procedures, the surgeon must turn towards the holding arm and away from the surgical instrument currently being used in order to readjust the endoscope. The realignment therefore represents an additional step in the procedure and extends its duration. In addition, the known holding arms often limit the freedom and flexibility of the surgeon, so that the known holding arms for endoscopes are perceived by surgeons as more annoying than helpful.

### SUMMARY OF THE INVENTION

It is therefore an object of the invention to eliminate the aforementioned disadvantages of the prior art and, in particular, to develop a device with which an endoscope can be used for a longer period of time without having to forego the flexibility in adjusting the endoscope required for the procedure.

The object of the invention is achieved, in an example, with a handle for medical endoscopes with a proximal contact surface for the endoscope and with a distal contact surface for the endoscope, wherein the direction of extension of the proximal contact surface is oriented at an angle greater than 0° with respect to the direction of extension of the distal contact surface.

The object of the invention is further achieved by an endoscope system with a medical endoscope and with a handle according to the invention, wherein the handle is connected to the endoscope, in particular, in a detachable manner.

The invention is based upon the fundamental finding that a major cause of the fatigue symptoms mentioned during prolonged use of the endoscope is that the endoscope, due to its geometric shape, its center of gravity, and its weight, is not ideally positioned in the surgeon's hand. Endoscopes typically have an endoscope shaft that is oriented axially, i.e., from proximal to distal, and which, in addition to a lumen for a working channel through which surgical instruments can be inserted and moved to the surgical site, has additional lumens—for example, lumens for light and/or irrigation channels. With modern endoscopes, the visual inspection of the surgical site is typically no longer carried out directly by the human eye; instead, the image information of the surgical site is digitally captured by modern endoscopes and presented to the surgeon on a display unit. For this purpose, a fiber optic connection is usually established in the form of an extension to the endoscope, via which the image information about the surgical site can be transmitted to the display unit by means of a camera cable. The fiber optic connection usually extends at an angle other than 0° from the endoscope shaft.

Endoscopes designed in this way cannot be gripped, moved, and held in an ergonomically optimal manner. This leads to fatigue, especially during longer procedures. In addition, due to the additional flushing connections of the endoscopes, the center of gravity of the endoscope does not rest ideally in the surgeon's hand. This also leads to accelerated fatigue. These problems are further exacerbated by the fact that surgeons have different hand sizes and shapes, so that no endoscope is optimally designed for all hand sizes. Thanks to the handle according to the invention, the flexibility guaranteed when using endoscopes manually does not have to be sacrificed. At the same time, the handle according to the invention enables more comfortable, ergonomic holding, guiding, and adjusting of the endoscope. Since the handle according to the invention can be individually manufactured for different hand sizes and shapes without great effort, it is in a certain sense also suitable as an adapter for endoscopes. The design of the proximal contact surface and the distal contact surface ensures that not only the endoscope shaft, but also the fiber optic connection rests comfortably in the surgeon's hand. The handle according to the invention therefore enables the surgeon to handle the endoscope ergonomically and prevents fatigue even during prolonged use.

For the purposes of the invention, proximal may refer to a direction towards the surgeon, and distal may refer to a direction towards the patient. In this respect, the direction of extension of a contact surface refers to its axial component. For the purpose of the invention, a proximal direction points forwards, while a distal direction points backwards.

The handle according to the invention can be grasped by the surgeon in, in particular, two different grip positions. In a first grip position, the pollex and the index of the surgeon enclose the endoscope shaft, i.e., they are oriented parallel to the direction of extension of the distal contact surface of the handle, while the surgeon's hand can rest on the proximal contact surface in the area of the os metacarpale pollicis, the metacarpal bone of the thumb. The surgeon's digitus anularis and digitus minimus enclose the handle in the area of the proximal contact surface. If necessary, the surgeon's digitus medius can also be placed on the endoscope shaft so that a trigital grip of the endoscope shaft is formed, which is also called a tridigital fingertip grip. In a second grip position, the handle according to the invention is enclosed by the surgeon's index in the region of the distal contact surface, whereby the digitus medius, the digitus annularis, and the digitus minimus are not in contact with the handle. The pollex rests on the proximal contact surface of the handle. Alternatively, the digitus medius can enclose the handle, and the index also rests on the proximal contact surface of the handle. In a standing hand position, the proximal contact surface of the handle extends away from the surgeon's hand, wherein the endoscope shaft extends—distally—downwards, away from the hand. During procedures on the spine, the second grip position is, in particular, suitable when the patient is in a prone position, and the surgeon wants to guide the endoscope perpendicularly to the spine.

The handle can be made in one piece and is therefore particularly easy to manufacture. The handle can also be designed in such a way that it can be operated left-handed and/or right-handed. For example, the handle is designed to be symmetrical to a plane of symmetry that is spanned by the axial direction and the vertical, and runs through the middle of the handle.

Preferably, the angle between the direction of extension of the proximal contact surface and the direction of extension of the distal contact surface is between 10° and 90°, in particular between 10° and 80°, more preferably between 15° and 75°, in particular between 30° and 60°, most preferably 45°.

In an advantageous development of the invention, at least one middle contact surface for the endoscope can be arranged between the proximal contact surface and the distal contact surface, wherein the middle contact surface can be in direct contact with the proximal contact surface and/or with the distal contact surface. The direction of extension of the middle contact surface can form an angle relative to the direction of extension of the distal contact surface between 10° and 90°, in particular between 10° and 80°, more preferably between 15° and 75°, in particular between 30° and 60°, most preferably 45°.

The proximal contact surface can have a depression in order to create a defined contact position for the endoscope, in particular for its fiber optic connection. At the same time, this prevents the endoscope from slipping. The depression may have at least one directional component that is axially aligned, and/or may be designed as a groove and/or as a bore. In order to further secure the endoscope against slipping, the depression of the proximal contact surface may have a cross-section that varies, particularly in the axial direction. This makes it, in particular, possible for the endoscope to be inserted into the depression with a tight fit, at least in sections.

The proximal contact surface may have at least one lateral extension to optimize the center of gravity of the handle to improve ergonomics. The extension may have an outer contour, in particular in the shape of a circular arc, wherein the outer contour may be ergonomically shaped. The outer contour can be adapted to a finger and/or a hand of the surgeon and serve as a contact surface for this. In a particularly advantageous embodiment, the proximal contact surface has an extension on both sides to enable right-handed as well as left-handed operation of the handle.

The proximal contact surface can have at least one undercut, which is arranged, for example, at a proximal region of the proximal contact surface. Particularly preferably, the undercut can be arranged in a proximal end region of the proximal contact surface. The undercut serves as a support for the endoscope and prevents the endoscope from slipping proximally—in particular, from slipping out of the handle.

The distal contact surface can have at least one depression with, in particular, a directional component in the axial direction. The depression can be oriented parallel to the axial direction. The depression of the distal contact surface may have a cross-section that is at least partially circular and/or may be designed to prevent the endoscope from slipping sideways in the region of the distal contact surface. It may be provided that the depression have a constant cross-section over its entire length. In a further embodiment of the invention, the width of the depression, in particular its cross-section, may be tapered from proximal to distal.

The distal contact surface may in a distal region have at least one protrusion which serves as an abutment for the endoscope, so that slipping of the endoscope distally relative to the handle is prevented. Particularly in combination with the undercut of the proximal contact surface, the protrusion of the distal contact surface serves to ensure that the handle can be positively connected to the endoscope, at least in the axial direction. The protrusion may extend axially over at least part of the axial length of the distal contact surface in order to reduce the risk of breakage of the protrusion under mechanical stress on the handle. For example, the protrusion extends over an axial length of about 10 mm to 20 mm, in particular 13 mm to 15 mm.

The protrusion can be designed as a partially annular extension and/or to be integral with the handle. The protrusion of the distal contact surface may extend over at least 180° in the circumferential direction in order to obtain a positive connection of the endoscope with the handle.

The protrusion can be designed as an annular extension. For example, it is provided that the inner diameter of the extension corresponds to the diameter of the depression of the distal contact surface, so that the endoscope can be arranged in a space which is delimited by the distal contact surface and the extension. In combination with the annular extension, the endoscope may be surrounded by the extension of the handle over its entire circumference, especially in the area of the endoscope shaft, so that the endoscope cannot fall out of the handle. This allows the handle to be positively connected to the endoscope.

The protrusion can have at least one, in particular two, depressions, wherein the at least one depression may be axially oriented in order to improve the reception of the endoscope in the handle. In particular, if the endoscope has lateral flushing connections, which are designed, for example, as Luer-Lock connections, these flushing connections can engage in the depressions of the protrusion, in particular, in the depressions of the annular extension. The depressions, which may be designed as through-openings, are formed as recesses, for example, and serve to improve the reception of the endoscope in the handle. In addition, the depressions enable the endoscope to be mounted in a rotationally fixed manner, in particular, with respect to an axially oriented axis of rotation.

The distal contact surface may have in a proximal region at least one, in particular lateral, projection, for example, in a transition region to the middle contact surface. The projection can be oriented perpendicular to the axial direction and serves, in particular, as an abutment for the endoscope in order to prevent the endoscope from slipping proximally. In particular, the at least one projection serves as an abutment for the flushing connections which, in some embodiments of the endoscopes, are arranged laterally on the endoscope and at an angle other than 0° to the axial direction. Preferably, as an abutment, a projection can be arranged on both sides of the depression of the distal contact surface.

The proximal contact surface may be designed to contact and/or receive a fiber optic connection and/or a camera connection of an endoscope.

The at least one middle contact surface can have, in particular, at least one depression for the endoscope, wherein the at least one depression of the at least one middle contact surface may have a smaller diameter than the diameter of the depression of the distal contact surface. The at least one middle contact surface may be designed to contact and/or receive a fiber optic connection and/or a camera connection of an endoscope. The distal contact surface can be designed to contact and/or receive an endoscope, in particular an endoscope shaft.

The handle may at its distal end region, in particular at its distal end face, have a projection the direction of extension of which has a directional component that is oriented perpendicularly to the axial direction. The protrusion of the distal front end may thus serve as an abutment for a finger of the surgeon, e.g., the index, and improves the ergonomics of the handle. In a further embodiment of the invention, it may be provided that the projection be obliquely oriented rear-proximally. In order to improve the ergonomics of the handle, its rear side may have a parabolic outer contour at least in some areas, wherein it is, in particular, provided that the apex of the outer contour be arranged at the axial height of the distal contact surface. In the region of the apex, the handle can receive a depression, directed towards the distal contact surface, which depression is, in particular, provided with an arcuate cross-section and/or is arranged in a ring shape.

To avoid damage to the endoscope, the material of the handle can be selected so that its hardness is lower than the hardness of the endoscope. The material can be sterilized, in particular autoclaved. In addition, the material of the handle may be biocompatible and have at least one component made of a thermoplastic and/or a homopolymer. The handle can be formed of or consists of polyamide-12, also referred to as PA12 or polylaurylactam. Furthermore, it may be provided that the roughness of the handle be less than 12 μm, wherein the roughness corresponds to the mean roughness value Ra, the quadratic roughness R_{q}, or the average roughness depth R_{z}. In addition, it may be provided that the weight of the handle be less than 500 g, in particular less than 200 g, preferably less than 150 g, most preferably less than 100 g. The handle may be designed as a single-use instrument or to be reusable. Preferably, the handle is designed to be symmetrical to an axis of symmetry, which comprises the axial direction.

The endoscope of the endoscope system may be positively or non-positively connected to the handle; in particular, it may be clampable thereto and/or lockable therewith. Preferably, the endoscope has at least one optical output and/or at least one flushing connection, which, for in the sense of the invention, creates additional locking options for the endoscope. The endoscope may be designed as an orthopedic endoscope, in particular for performing transforaminal and/or interlaminar and/or intradiscal and/or cervical procedures on the spine. The endoscope can be designed as a foraminoscope and/or as a laminoscope and/or as a nucleoscope.

The endoscope may be in direct contact with the proximal contact surface and with the distal contact surface of the handle. It may be provided that a fiber optic connection and/or a camera connection of the endoscope rest on the proximal contact surface of the handle and/or be received by it. Furthermore, it may be provided that the fiber optic connection and/or the camera connection of the endoscope rest on and/or be received by the at least one middle contact surface of the handle. Furthermore, it may be provided that the endoscope, in particular its endoscope shaft, rest on the distal contact surface of the handle and/or be received by it.

Further scope of applicability of the present invention will become apparent from the detailed description given hereinafter. However, it should be understood that the detailed description and specific examples, while indicating preferred embodiments of the invention, are given by way of illustration only, since various changes, combinations, and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limitive of the present invention, and wherein:

FIG. 1 shows a handle according to the invention in a perspectival side view,

FIG. 2 shows the handle according to FIG. 1 in a side view rotated by 180°,

FIG. 3 shows the handle according to FIG. 1 in a frontal view,

FIG. 4 shows the handle according to FIG. 1 in a side view,

FIG. 5 shows the handle according to FIG. 4 in a view rotated by 180°,

FIG. 6 shows an enlarged view of the distal region of the handle of FIG. 1,

FIG. 7 shows the handle according to FIG. 1 in a view from below,

FIG. 8 shows the handle according to FIG. 7 in a view rotated by 180°,

FIG. 9 shows an endoscope system with the handle according to FIG. 2 and an endoscope connected thereto,

FIG. 10 shows the endoscope system of FIG. 9 in a frontal view,

FIG. 11 shows the endoscope system of FIG. 9 in a view from the rear,

FIG. 12 shows the endoscope system of FIG. 9 in a side view, and

FIG. 13 shows the endoscope system of FIG. 13 in a reduced view.

### DETAILED DESCRIPTION

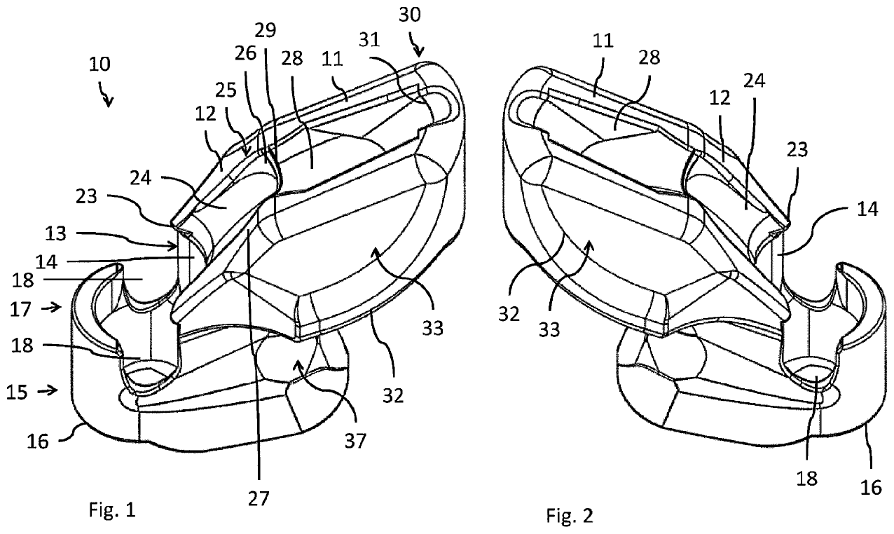

FIG. 1 shows a perspectival side view of a handle 10 according to the invention for a medical endoscope 39, wherein the handle 10 has a proximal contact surface 11, a middle contact surface 12, and a distal contact surface 13. In the sense of the invention, a proximal direction corresponds to a direction pointing towards the user, and a distal direction corresponds to a direction pointing towards the patent. The direction of extension from proximal to distal corresponds to an axial direction. The middle contact surface 12 is arranged between the proximal contact surface 11 and the distal contact surface 13.

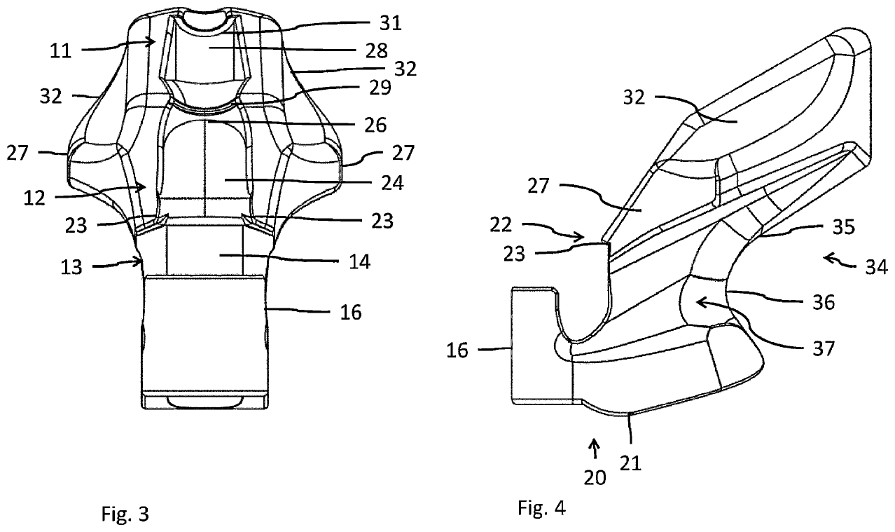

The direction of extension of the distal contact surface 13 of the handle 10 according to FIG. 1 is oriented parallel to the axial direction, so that an endoscope shaft 40 can be arranged in axial orientation on the distal contact surface 13. In order to stabilize the endoscope shaft 40 on the distal contact surface 13, the latter over its entire length has a depression 14 with a partially circular cross-section, which is constant over the entire length of the distal contact surface 13. In the exemplary embodiment shown, the depression 14 is designed as a groove. This is also shown in the perspectival view of FIG. 2 and in the frontal view according to FIG. 3. The design of the handle 10 is symmetrical to a plane of symmetry which includes the axial direction which is oriented parallel to the direction of extension of the handle 10. Therefore, the illustration in FIG. 2 essentially corresponds to the illustration in FIG. 1.

In a distal region 15, the distal contact surface has an annular extension 16 with an axial length of approximately 10 mm to 20 mm, in order to prevent the endoscope shaft 40 from falling out of the handle 10. The extension 16 is hollow-cylindrical and axially oriented. In the exemplary embodiment shown, the inner diameter of the extension corresponds to the diameter of the depression 14 of the distal contact surface 13.

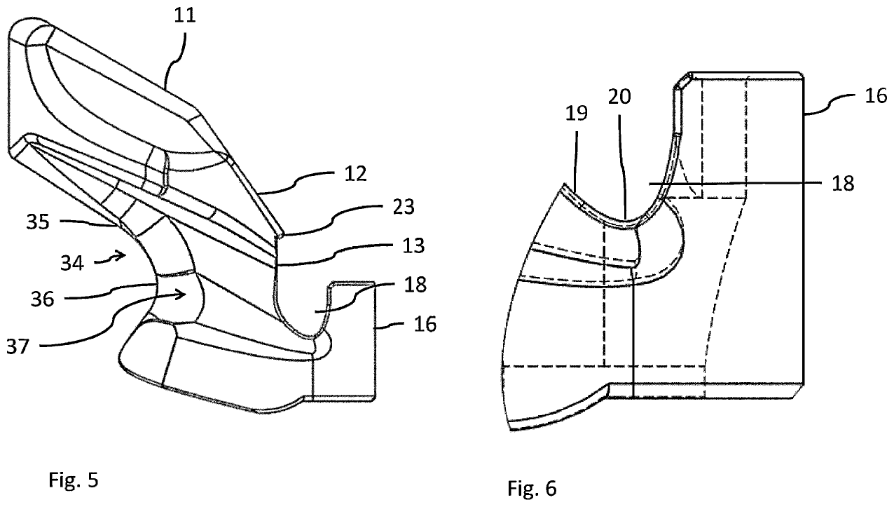

According to FIGS. 1 and 2, in a proximal region 17 of the extension 16, two oppositely disposed, elongated, hole-shaped recesses 18 are formed as depressions, which serve to receive Luer-Lock connectors arranged laterally of the endoscope, e.g., flushing connectors 42, and can ensure additional fixation of the endoscope 39 in the sense of an anti-rotation lock. From the side views of FIG. 4 and FIG. 5 and, in particular, from the enlarged side view of the depressions 18 of the extension according to FIG. 6, it is clear that the outer contours 19 of the recesses 18 are each parabolic, and that a stepless transition of the recesses to the one in FIG. 6 on the left side of the distal contact surface 13 is formed.

In a distal end region 20, the distal contact surface 13 has a projection 21 which extends downwards in the proximal direction and forms the distal front end of the handle 10. The distal surface of the projection 21 obliquely extends rear-proximally according to the side views of FIG. 4 and FIG. 5, in order to serve as an abutment for a finger of the surgeon. Due to the already mentioned axially symmetrical design of the handle 10, the illustration in FIG. 5 corresponds to the illustration in FIG. 4.

The distal contact surface 13 has, in a proximal region, two lateral projections 22 which lie opposite one another and which serve as abutments for the endoscope 39 and prevent the endoscope from falling out proximally.

The middle contact surface 12 directly adjoins the distal contact surface 13 in the proximal direction, wherein the direction of extension of the middle contact surface 12 is arranged at an angle of 35° relative to the direction of extension of the distal contact surface 13. The middle contact surface 12 also has a partially circular depression 24, which is designed as a groove in such a way as to accommodate a fiber optic connection 41 or camera connection of the endoscope 39. The diameter of the depression 24 of the middle contact surface 12 is therefore smaller than the diameter of the depression 14 of the distal contact surface 13. In a proximal region 25, the middle contact surface 12 is provided with an undercut 26 in the region of its depression 24 in order to prevent the endoscope 39 from accidentally falling out.

On both sides of the depression 24 of the middle contact surface 12—thus perpendicular to the axial direction—an extension 27 is formed on the central contact surface 12, which serves as a finger rest when the handle 10 is used.

The middle contact surface 12 merges proximally into the proximal contact surface 11. The direction of extension of the proximal contact surface 11 is arranged at an angle of 25° relative to the direction of extension of the middle contact surface 12, and thus at an angle of 60° relative to the direction of extension of the distal contact surface 13. The proximal contact surface 11 has a depression 28, wherein, in particular according to FIG. 1 to 3, between the depression 24 of the middle contact surface 12 and the depression 28 of the proximal contact surface 11, a protrusion 29 is formed, which serves as an abutment for the endoscope 39.

The diameter of the depression 28 of the proximal contact surface 11 is variable over its length in order to accommodate the endoscope 39 with a tight fit. In a proximal region 30 of the proximal contact surface 11, the depression 28 of the proximal contact surface 11 has an undercut 31 which serves as an axial abutment for the endoscope 39 and prevents the endoscope 39 from falling out proximally.

On both sides of the depression 28 of the proximal contact surface 11, extensions 32 extending perpendicular to the axial direction are formed on the proximal contact surface 11 and serve as finger rests for the user. For ergonomic reasons, the extensions 32 each have a circular-arc-shaped upper outer contour 33.

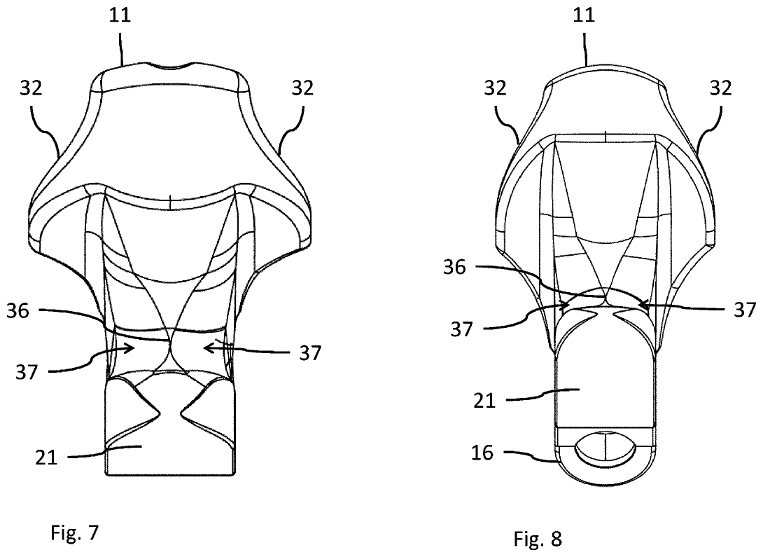

According to the side views of FIGS. 4 and 5, the rear side 34 of the handle 10 has an approximately parabolic outer contour 35, the apex 36 of which is arranged at the axial height of the distal contact surface 13, so that the endoscope 39 can be held ergonomically by the surgeon. In the area of the apex 36, the handle 10 has an annular depression 37 with an arcuate cross-section and oriented towards the distal contact surface 13, in order to improve the ergonomics of the handle 10. This is also evident from the proximal rear view of the handle 10 according to FIG. 7, as well as from the view rotated by 180° according to FIG. 8.

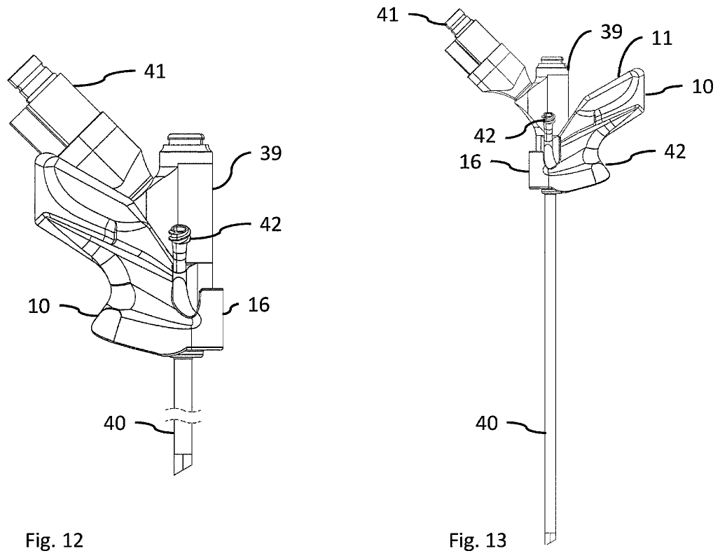

In particular, two grip positions of the handle 10 are possible and are explained with reference to the illustration according to FIG. 4, wherein the endoscope shaft 40 is arranged vertically and passes through the annular extension 16 of the distal contact surface 13. The fiber optic connection 41 of the endoscope 39 rests on the middle contact surface 12 and on the proximal contact surface 11, and extends upwards to the right, as shown in FIG. 4. In the first grip position, for example, the right hand of the surgeon approaches the handle 10 according to FIG. 4 from above, i.e., proximally, wherein the index and the pollex are in contact with the endoscope shaft 40, i.e., are axially oriented. The surgeon's hand rests in the area of the os metacarpale pollicis on the lateral extension 32 of the proximal contact surface 11. If necessary, the index can detach from the endoscope shaft 40 and engage around the distal end region 20 of the handle 10, the distal projection 21 of the distal contact surface 13, as an abutment. The arrangement of the endoscope 39 on the handle 10 connected to it in the first handle position is shown in FIG. 12.

In the second grip position, for example, the left hand of the surgeon encloses the handle 10 coming from the left as shown in FIG. 4. The surgeon's index encloses the apex 36 of the outer contour 35 of the rear side 34 of the handle 10, while the pollex rests on the endoscope shaft. The arrangement of the endoscope 39 on the handle 10 connected to it in the second handle position is shown in FIG. 13.

The handle 10 according to the invention is designed to be symmetrical to a plane of symmetry that is spanned by the axial direction and the vertical, and runs through middle of the handle 10. As a result, the handle 10 according to the invention can be operated both left-handed and right-handed, in particular, with regard to the handle positions described above.

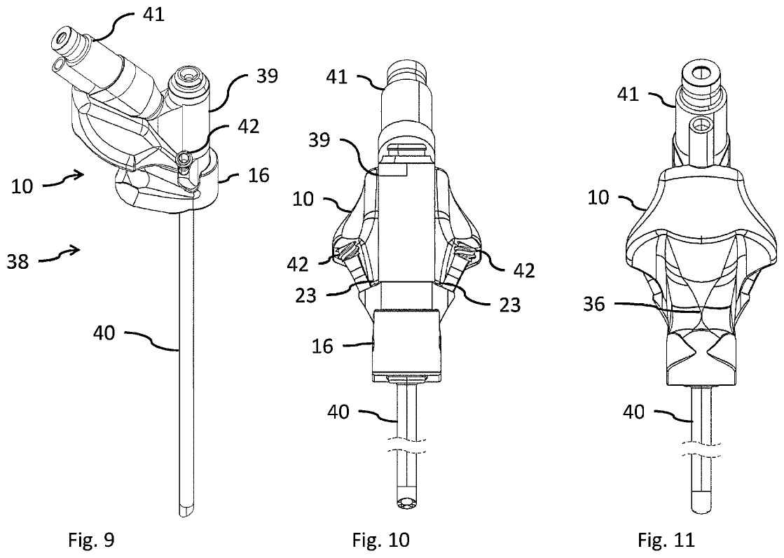

FIG. 9 shows an endoscope system 38 according to the invention with the handle 10 according to the invention and the endoscope 39 detachably connected thereto such that the endoscope 39 is in contact with the proximal contact surface 11, the middle contact surface 12, and the distal contact surface 13 of the handle 10. The fiber optic connection 41 or camera connection of the endoscope 39 engages in the depression 28 of the proximal contact surface 11 and is oriented at an angle of approximately 60° relative to the axially oriented direction of extension of the endoscope shaft 40. The endoscope 39 engages in the depression 14 of the distal contact surface 13 of the handle 10 and passes through the annular extension 16. The endoscope 39 is provided with flushing connections 42, each of which is arranged at an angle of greater than 0° relative to the direction of extension of the endoscope 39, and thus also relative to the endoscope shaft 40, and which are each designed as a Luer-Lock connection, which is also evident from the frontal view of the endoscope system 38 in FIG. 10.

The flushing connections 42 are each in contact with the projections 23 of the distal contact surface 13, so that a movement of the endoscope 39 relative to the handle 10 in the proximal direction is prevented. A movement of the endoscope 39 relative to the handle 10 in the distal direction is prevented by the flushing connections 42 of the endoscope 39 passing through the depressions 18 of the annular extension 16. FIG. 11 shows the endoscope system 38 in a rear view.

FIG. 12 shows the endoscope system 38 according to FIG. 9 in a side view, wherein the handle 10 is in the already described first handle position in which the fiber optic connection 41 of the endoscope 39 is in contact with the proximal contact surface 11 of the handle 10. FIG. 13 shows the endoscope system 38 according to FIG. 9 in the second handle position, also already described, in which the fiber optic connection 41 is not in contact with the proximal contact surface 11 of the handle 10. In the second handle position, the endoscope 39 is rotated by 180° around its direction of extension, compared to the first handle position. In both grip positions, the endoscope shaft 40 penetrates the annular extension 16, and the flushing connections 42 are in contact with the projections 23 on the one hand and pass through the depressions 18 on the other, so that a reliable, positive connection of the endoscope 39 with the handle 10 is ensured.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

What is claimed is:

1. A handle for medical endoscopes, the handle comprising:
a proximal contact surface for the endoscope;
a distal contact surface for the endoscope, wherein a direction of extension of the proximal contact surface is oriented at an angle greater than 0° with respect to a direction of extension of the distal contact surface (13).

2. The handle according to claim 1, wherein the angle between the direction of extension of the proximal contact surface and the direction of extension of the distal contact surface is between 10° and 90°, or is 45°.

3. The handle according to claim 1, wherein at least one middle contact surface for the endoscope is arranged between the proximal contact surface and the distal contact surface.

4. The handle according to claim 1, wherein the proximal contact surface has a depression.

5. The handle according to claim 4, wherein the depression of the proximal contact surface has a variable cross-section.

6. The handle according to claim 1, wherein the proximal contact surface has at least one lateral extension, and wherein the extension has an arcuate outer contour.

7. The handle according to claim 1, wherein the proximal contact surface has an undercut in a proximal region.

8. The handle according to claim 1, wherein the distal contact surface has a depression with an at least partially annular cross-section.

9. The handle according to claim 1, wherein the distal contact surface has a protrusion in a distal region, and wherein the protrusion is designed to be at least partially annular.

10. The handle according to claim 9, wherein the protrusion extends in a circumferential direction over at least 180°.

11. The handle according to claim 9, wherein the protrusion is designed as an annular extension, wherein an inner diameter of the extension corresponds to a diameter of the depression of the distal contact surface.

12. The handle according to claim 9, wherein the protrusion has at least one depression or two depressions, and wherein the at least one depression is axially oriented.

13. The handle according to claim 1, wherein the distal contact surface has at least one lateral projection in a proximal region.

14. The handle according to claim 1, wherein the handle has at its distal end region a projection, a direction of extension of which has a directional component which is oriented substantially perpendicular to the axial direction.

15. An endoscope system comprising:
a medical endoscope; and
the handle according to claim 1, the handle being detachably connected to the endoscope.

16. The endoscope system according to claim 15, wherein the endoscope is in contact with the proximal contact surface and with the distal contact surface of the handle.
